(19) United States
(12) Patent Application Publication
(10) Pub. No.: US 2003/0129248 A1
(43) Pub. Date: Jul. 10, 2003

(54) Microcapsule, in particular, for immobilizing organic or inorganic solids, liquids and/or gases

(76) Inventor: Rainer Pommersheim, Mainz (DE)

Correspondence Address: Andrus Sceales Starke & Sawall, Suite 1100, 100 East Wisconsin Avenue, Milwaukee, WI, 53202-4178, US

(21) Appl. No.: 10/220,464

(22) PCT Filed: Feb. 16, 2001
(86) PCT No.: PCT/EP01/01743

(30) Foreign Application Priority Data
Feb. 29, 2000 (DE) 100 09 440.6

(51) Int. Cl. A61K 9/16; A61K 9/50

(52) U.S. Cl. 424/490

(57) ABSTRACT

The invention relates to a microcapsule, in particular, for immobilizing organic or inorganic solids, liquids and/or gases, which is to be used in the fields of food engineering, pharmaceuticals and/or chemistry. The inventive microcapsule comprises a preferably spherical core containing the substance to be immobilized and comprises a covering that encloses said core. According to the invention, a phase limit that prevents the diffusion of small molecules is formed out of two substances, which are preferably liquid and which cannot be mixed with one another, inside the capsule or in the capsule covering.

### DESCRIPTION

The invention relates to a microcapsule, in particular for immobilizing organic or inorganic solids, liquids and/or gases according to the precharacterizing clause of claim 1, such that even living cells or microorganisms can constitute solid materials in the sense of the invention.

In technological practice as well as in medicine it is often necessary to immobilize solid materials, but also liquids and/or gases. This requirement can arise for purely economical reasons, because by this means expensive agents can be reclaimed, but it can also be imposed by process technology because it enables sensitive additives to be protected from the surrounding medium.

For example, in food technology it can happen that substances sensitive to oxygen and/or moisture are added to certain products. If these additives are not protected from the surrounding medium, which as a rule is oxygen-rich and/or moist, they become oxidized with the result that the products will keep for a considerably shorter time. Such additives can, for example, be artificial aromas or solids such as iron, fillers, microorganisms etc. To ensure that these additives remain up to standard until the use-by date of the food, either the time until the date is made relatively short or the substances are added in correspondingly higher dosages.

In other cases, for instance, substances must be added to media with which they react, which would cause them to be destroyed. Therefore it is desirable for such substances to make contact with the ambient media only after some delay, i.e. just before the mixture is used, so as to ensure their maximal efficiency. Such additives include, e.g., some agents contained in cosmetics, which become active only when they contact the skin, but they can also be aromas that are liberated only when the food containing them is chewed.

At several places in the relevant literature microcapsules are described that serve to immobilize a great variety of additives. For example, the unexamined application DE 196 44 343 A1 describes a microcapsule having neutral taste, with a diameter of a few μm, which is produced in an emulsion process and can be used as an addditive in food products or animal feed, and can also serve as a transport system for medicines. In this case oils, or substances soluble in this oil, are emulsified in a base material such as alginate, and from this mixture, in another emulsion process, 0.5- to 20-μm capsules are formed, which can then be employed in the food or pharmaceutical industry. However, these small spheres are unsuitable for immobilizing relatively large solid particles such as granulates, nor can they be employed, for instance, in citrate-containing media because citrate would destroy the alginate shell of these capsules.

In the patent U.S. Pat. No. 4,389,419 a similar method is described for the encapsulation of oils and oil-soluble substances. As in the protective right cited above, here an emulsion of the oil with a base material (alginate) is produced in a first step. In this case, however, certain fillers are also added to the alginate and the capsules are shaped by extrusion through a nozzle and precipitation in a precipitation bath, rather than by an additional emulsification step. These capsules are larger than those described in the first citation, but they are likewise unsuitable for use in citrate-containing media. Furthermore, part of the encapsulated oil will bleed out of the capsule under a high mechanical load, as though from an oil-saturated sponge.

In a class of their own are the so-called membrane capsules. F. Lim and A. Sun published in the journal Science Vol. 210, pp. 909-910, year 1980, a description of a capsule with a semipermeable membrane for immobilizing living cells, in which the core of the capsule is surrounded by a single layer of a poly-L-lysine/alginate complex. In these capsules the cells are prevented from emerging out of the capsule core. This membrane capsule is unsuitable for use in technical processes because of its relatively slight mechanical stability. Furthermore, no molecules of the size of an enzyme or smaller can be enclosed therein, because the membrane is permeable to them.

In the document DE 43 12 970.6 A1 a membrane capsule is described that is also suitable for immobilizing enzymes and proteins. Here the core containing the substance to be immobilized is enclosed in a cover comprising several layers, each of which endows the cover as a whole with a particular property. By choosing the cover polymers appropriately, the permeability of the membrane can be reduced to such an extent that the enzymes remain in the capsule, whereas the much smaller substrates and products can pass through the membrane. These capsules, however, are entirely unsuitable for immobilizing small molecules, which cannot be retained by the membrane.

The objective of the invention is thus to disclose a capsule that is impermeable to small molecules, that can be employed in a great number of media and is also suitable for use in the food and pharmaceutical industries, and that can be dried and stored in a simple manner.

This objective is achieved with an object according to claim 1, while the subordinate claims comprise at least advantageous embodiments and further modifications.

The capsule in accordance with the invention contains in its interior and/or in its cover a barrier to small molecules such as oxygen and other gases. At the same time its mechanical stability can be adjusted such that it can be employed in technical processes and/or destroyed by simple mechanical actions (application to the skin). The capsule can be used in a large variety of media, which it does not contaminate by bleeding-out, and can also be dried with no sacrifice of its function.

In accordance with the invention a capsule is provided in the interior and/or membrane of which a barrier to small molecules is formed by the interaction of two mutually immiscible liquids, one of which for example is water or an aqueous solution. The other liquid can, for instance, be an oil or another substance immiscible, e.g., with water such as a carbohydrate, a carbohydrate mixture and/or solutions of various substances in carbohydrates.

The central idea of the invention thus resides in producing a two-phase system in the interior of the capsules and/or on the capsule surface, such that the immobilized substance is soluble either in only one phase or in neither of the two plases, and the phase in which it is insoluble always completely encloses it.

By this means it is possible to encapsulate solid materials as well as liquids and/or gases, but also living objects such as cells and bacterial cultures; here the substance to be immobilized is advantageously contained in the phase that is not miscible with water. This can be achieved by a suspension in the case of a solid material, or by an emulsion or solution in the case of a liquid or a gas.

Accordingly, this capsule is constructed as follows: its core consists of a base material from which a matrix is formed in which the substance to be immobilized, surrounded by a liquid immiscible with the matrix material, is embedded. This base material must be a substance capable of being dispensed as droplets and/or emulsified, from which by means of precipitation under the action of ions or a temperature gradient preferably spherical particles can be formed. Such substances include, for instance, sodium alginate but also agarose or Sephadex as well as paraffins or ceramics etc.

If the liquid immiscible with the matrix material, which encloses the immobilized substance, is a low-viscosity oil or a volatile hydrocarbon or in all other cases such that the capsule is to be formed with a particular mechanical stability, it is advantageous to enclose the capsule core in an additional membrane. This membrane can consist of a polyelectrolyte complex, which can be applied in multiple layers. Polyelectrolyte complexes of this kind are formed by the interaction of a polyanion and polycation. As the polyanion, water-soluble cellulose derivatives such as carboxymethyl cellulose, cellulose sulfate or pectins, alginates as well as synthetic polymers such as polyacrylic or polymethacrylic acids and the like are advantageously employed. Chief among the polycations to be considered are natural substances such as chitosan, but also synthetic polymers such as polyethylene imine or polydiethyl diallyl ammonium chloride.

However, a membrane on the capsule surface can also be produced by drying. This can be done in two ways. Either the capsules are incompletely dried, for instance with relatively hot air, so that a crust forms on its surface, or other substances are blown into the dryer along with the drying air. These preferably solid or liquid substances become firmly attached to the capsule surface and thus form a membrane; this situation is referred to as “coating”. If the capsules are to be employed in the food or pharmaceutical area, these coating substances can be sugar, milk powder, flour, shellac, alginate or another substance permitted for the particular purpose. For chemical purposes other, preferably film-forming compounds such as nitrocellulose derivatives or polyvinyl acetate etc. can also be used.

Another approach in accordance with the invention is to incorporate the two-phase system and hence the barrier not into the core of the capsule but rather into its membrane. In this case, again, the capsule core consists of a substance that can be dispensed as droplets and from which preferably spherical particles can be formed by means of precipitation due to ion action or a temperature gradient. The substance to be immobilized, however, is directly dissolved or suspended in this matrix substance. Such substances can, as in the first case, be e.g. sodium alginate but also agarose or Sephadex or even paraffins or ceramics etc.

The membrane that completely encloses the capsule core can be applied to the core either as already described above, by precipitation of polyelectrolytes onto the capsule surface, or by a later coating process. In contrast to the above-mentioned capsule, here in every case a layer must be present that is immiscible with the under- and/or overlying layer, so that a phase boundary is created in the membrane. Such a layer can be produced, for example, by oils or fats or also by low-molecular-weight or macromolecular carbohydrates. So that this layer can be anchored to the under- and overlying layers, it is advantageous for the substance to contain polar groups.

In some cases it can also be advantageous to produce a capsule by a combination of the two above-mentioned approaches. Such a capsule would then have its diffusion barriers not only in the core but also in the capsule membrane, which would enhance its reliability.

For certain applications, for instance in cosmetics, it is essential for the capsule to have a particular degree of mechanical stability, i.e. to be destroyed and release the agent it contains only when the imposed load reaches a particular level. This can be achieved on one hand by an appropriate selection of the polymers and the number of layers in the cover, but on the other hand in many cases the matrix of the capsule core can be destroyed after the covering has been applied, in an additional procedural step that makes it fluid again. The overall mechanical stability in this case is provided to the capsule only by its cover. This can happen, for example, when Na alginate is chosen as the matrix material and it gels by precipitation in a solution of a polyvalent metal ion. This gelation can be reversed after the covering has been applied, by exposing the capsule to a Na citrate solution.

In the following the invention is explained in greater detail with reference to an exemplary embodiment.

A method of producing the microcapsule, for example one that is to be used in the food industry in order to protect metallic iron from oxidation in a moist medium, proceeds as follows. First iron powder is suspended in a small amount of edible oil, such as olive oil. This iron/oil suspension is subsequently emulsified in a relatively large amount of Na-alginate solution. In a further step this emulsion can be dripped by means of suitable apparatus into a precipitation bath containing a multivalent metal ion, as a result of which relatively large particles are formed. However, the emulsion can also be stabilized and/or precipitated directly by adding to it a solution of a multivalent metal ion, in which case particles in the μm range are produced.

In this way an alginate matrix is formed that surrounds the oil-enclosed iron. Because the oil is immiscible with the aqueous alginate, a diffusion barrier is simultaneously formed around each iron particle. If a small amount of a polyelectrolyte is additionally mixed into the precipitation bath, a thin membrane that prevents bleeding-out of superfluous oil from the capsule is formed at the same time as the precipitation occurs. By repeatedly exposing this capsule to differently charged polyelectrolyte solutions, a membrane can be built up that endows the capsule with a degree of mechanical stability appropriate to the application. For capsules with very small diameters, for example less than 100 μm, it is advantageous for this exposure to take place in fluidized-bed form. For this purpose, the capsules are placed in a vessel such that the coating solutions can flow past them at a velocity great enough not only to swirl them around but also to keep them suspended.

Although in some cases the capsule can also be employed without a cover, an additional cover is advantageous. Such a cover can be made of polyelectrolyte complexes, but can also be applied by a coating process. It is also advantageous to use a combination of the two covers, one complexed and one applied by coating.

In accordance with the invention the coating can be done in such a way that while the capsules are being dried in a turbulent stream, after they have lost some of their moisture a solid material in powder form is blown into the drying column and adheres to the capsules so as to enclose them completely. A material suitable for this purpose, for example, is milk powder. If the drying is continued until the capsules contain a small residual moisture, the result is that each capsule is surrounded by a crust of dried solid material, e.g. milk. The capsules formed in this way are white in color and can be stored for months with no oxidation of the iron they contain, even in a relatively moist environment.

The two-phase system in the interior of the capsule can be implemented as described below. To produce droplets a nozzle is used that has in its interior two concentrically arranged capillaries. These capillaries are situated within a cylinder into which air is blown, which flows concentrically past the outer capillary and thus causes the droplet to break off cleanly.

In order to encapsulate, e.g., an enzyme that can be introduced into a liquid detergent concentrate under isosmopolar conditions, the procedure is as follows. First several solutions are prepared. Solution A consists of the detergent concentrate, the enzyme and the precipitation reagent, e.g. CaCl₂, BaCl₂ and sometimes polycation or polyanion, but in every case a polymeric counterion to the base material. The base material comprises a solution of, e.g., Na alginate, Sephadex, agarose etc. The precipitation bath consists of, e.g. CaCl₂, BaCl₂ and sometimes polycation or polyanion, but in every case a polymeric counterion to the base material.

For the encapsulation, solution A is then pressed through the inner capillary of the nozzle and the base material, through the outer capillary. The concentric air current produces droplets that contain solution A in the interior, surrounded by base material. These droplets are gelled by dripping them into the precipitation bath. Thereafter the gel particles can be covered as described above.

In order to obtain greater stability in aggressive media, it is possible in a subsequent step to produce chemical cross-linkage of the applied layers, which can be done for instance with carbon diimides.

The capsules are stored in the detergent concentrate used to produce solution A, and when the concentrate is diluted, they break and release the enzyme. In this way several different enzymes can be introduced simultaneously into liquid detergent, which in the prior art has not been possible by simple means.

1. Microcapsule, in particular for immobilizing organic or inorganic solids, liquids and/or gases, to be used in food technology, pharmacy and/or chemistry, with a preferably spherical core that contains the substance to be immobilized and preferably a cover enclosing the core, characterized in that
in the interior of the capsule and/or in the cover of the capsule a phase boundary is formed by two mutually immiscible, preferably liquid substances.

2. Microcapsule according to claim 1, characterized in that
one of the mutually immiscible substances forming the capsule core forms a matrix, in the interior of which the second substance is distributed, such that the substance to be immobilized is completely enclosed by the second substance situated in the interior of the matrix.

3. Microcapsule according to claim 2, characterized in that
the substance to be immobilized is embedded in the interior of the matrix and/or of the cover.

4. Microcapsule according to one of the preceding claims, characterized in that
the capsule core is surrounded by a preferably multi-layered cover that completely encloses it.

5. Microcapsule according to claim 4, characterized in that
the cover enclosing the core contains at least one layer that is separated by a phase boundary from the adjacent under- and/or overlying layer, such that the former layer is impermeable to substances of low molecular weight.

6. Microcapsule according to claim 4 or 5, characterized in that
the cover contains at least one layer that is covalently and/or electrostatically connected to the adjacent under- and/or overlying layer.

7. Microcapsule according to claim 4 to 6, characterized in that
at least one layer of the cover is formed by drying the capsule surface.

8. Microcapsule according to one of the claims 4 to 6, characterized in that
at least one layer of the cover is formed by coating the capsule core with a solid substance.

9. Microcapsule according to one of the claims 4 to 6, characterized in that
at least one layer of the cover is formed by a membrane-forming fluid on the capsule core.

10. Microcapsule according to one of the claims 4 to 9, characterized in that
at least one layer of the cover is formed both by complex-formation and also by coating.

11. Microcapsule according to one of the preceding claims, characterized in that
the substance that forms the core matrix is a material that can be applied as droplets and/or can be emulsified, from which preferably spherical particles can be formed by means of precipitation under the action of ions or a temperature gradient.

12. Microcapsule according to one of the preceding claims, characterized in that
the material surrounding the substance to be immobilized is an oil or another liquid immiscible with the matrix material.

13. Microcapsule according to claim 12, characterized in that
the material is a carbohydrate or a carbohydrate mixture.

14. Microcapsule according to one of the claims 2 to 13, characterized in that
the substance to be immobilized forms a suspension with the second substance in the interior of the capsule, namely the material that forms a phase boundary with the matrix material.

15. Microcapsule according to one of the claims 2 to 13, characterized in that
the substance to be immobilized forms a liquid/liquid emulsion with the second substance in the interior of the capsule, namely the material that forms a phase boundary with the matrix material.

16. Microcapsule according to one of the claims 2 to 13, characterized in that
the substance to be immobilized forms a gas/liquid emulsion with the second substance in the interior of the capsule, namely the material that forms a phase boundary with the matrix material.

17. Microcapsule according to one of the preceding claims, characterized in that
the enveloping membrane is impermeable to substances of low molecular weight.

18. Microcapsule according to one of the preceding claims, characterized in that
the capsule core is impermeable to substances of low molecular weight.

19. Microcapsule according to one of the preceding claims, characterized in that
at least one phase in the capsule core contains a solid material.

20. Microcapsule according to one of the preceding claims, characterized in that
at least one phase in the capsule core contains a liquid.

21. Microcapsule according to one of the preceding claims, characterized in that
at least one phase in the capsule core contains a gas.

22. Microcapsule according to claim 1, characterized in that
one of the mutually immiscible substances that form the capsule core is completely enclosed by the second substance.

23. Microcapsule according to one of the claims 1 to 22, characterized in that
the process of covering the capsules is carried out by a fluidized-bed method.

24. Microcapsule according to one of the preceding claims, characterized in that
during drying a membrane or crust is produced on the capsule surface.

25. Microcapsule according to one of the claims 1 to 24, characterized in that
during drying by a fluidized-bed method a solid material is additionally blown into the column, so that a membrane is produced on the capsule surface.

26. Microcapsule according to one of the preceding claims, characterized in that
at least one phase in the capsule core contains living cells and/or microorganisms and/or one or more additives.
